(19) United States
(12) Patent Application Publication
(10) Pub. No.: US 2018/0050076 A1
(43) Pub. Date: Feb. 22, 2018

(54) Kung Fu Gu

(71) Applicant: James Russell Prine, Clermont, FL (US)

(72) Inventor: James Russell Prine, Clermont, FL (US)

(21) Appl. No.: 15/241,591

(22) Filed: Aug. 19, 2016

(51) Int. Cl. A61K 36/87; A61K 9/00; A61K 9/06; A61K 35/644; A61K 36/708; A61K 36/288; A61K 36/756; A61K 36/744; A61K 36/539; A61K 36/28; A61K 36/30; A61K 36/185; A61K 36/324; A61K 36/328; A61K 31/045; A61K 31/235; A61K 36/53; A61K 36/61; A61K 36/286

(52) U.S. Cl. CPC A61K 36/87; A61K 36/286; A61K 9/06; A61K 35/644; A61K 36/708; A61K 36/288; A61K 36/756; A61K 36/744; A61K 36/539; A61K 36/28; A61K 36/30; A61K 36/185; A61K 36/324; A61K 36/328; A61K 31/045; A61K 31/235; A61K 36/53; A61K 36/61; A61K 9/0014

(57) ABSTRACT

Kung Fu GU: a topical balm, circulatory, emollient, that helps accelerate the healing of Bruises, reduces swelling and pain as well as increasing circulation. Comprised of Grape seed oil, Bees wax, Rhubarb, Phellodendron, Safflower, Dandelion, Gardenia fruit, Skullcaps, Arnica Montana, Comfrey, Devil's claw, Frankincense, Myrrh, Dragon's Blood resin, Menthol crystals, Wintergreen oil, Birch oil, Lavender oil, Cajuput oil, Clove oil, and the method of preparing same said circulatory emollient balm.

### BACKGROUND OF THE INVENTION

The present invention relates to the recovery and healing of bruises strained or sprained tendon and ligaments more particularly pertains to a new invention that accelerates the healing of bruises and if used in a timely manner will help to prevent the manifestation of a bruise. As well as reducing pain and inflammation of strained or sprained tendon and ligaments, further more increases circulation which also expedites the healing process, additionally serving as an all-purpose massage circulatory, emollient balm.

### DESCRIPTION OF PRIOR ARTS

The use of herbs to help relieve pain and swelling is known in prior art however these have not included the synergetic healing properties of certain natural resins, as well as the plurality of essential oils, which are well known to have certain healing and muscle relaxing properties. For this reason such a composition is needed which is extremely well adapted to accelerate the healing of bruises, and if used in a timely manner will help to prevent the manifestation of a bruise. As well as reducing pain and inflammation of strained or sprained tendon and ligaments, furthermore increasing circulation which also expedites the healing process for this reason Kung Fu GU is well suited to also be used for general all-purpose massage circulatory purposes.

### DESCRIPTION OF PREFERRED EMBODIMENT

In order to produce Kung Fu GU, after obtaining the ingredients listed the following method should be employed.

Herbs: combine the following
1 oz. Rhubarb root
1 oz. Safflower
1 oz. Skullcaps
1 oz. Phellodendron
1 oz. Dandelion greens
1 oz. Gardenia fruit
1 oz. Devil's claw
1 oz. Comfrey root
1 oz. Comfrey leaf
2 oz. Arnica Montana

To define, Herbal mixture.

Position for mentioned Herbal mixture in a cooking vessel and cover with between 24 and 36 ounces of Grape seed oil.

The said Grape seed oil and herbal mixture are then heated to a temperature below the boiling point of the Grape seed oil until desire color and fragrance are achieved to define Infusion oil.

Strain Infusion oil through cheesecloth collect said strained infused oil to define Strained Infusion oil.

Resins: combine the following
2 oz. Frankincense resin
2 oz. Myrrh resin
2 oz. Dragon's blood resin

To define Resin Mixture.

Position the for mentioned Resin Mixture in a cooking vessel and cover with between 24 and 36 ounces of Grape seed oil said Grape seed oil and resin mixture are heated to a temperature below the boiling point of the Grape seed oil until desired color and fragrance are achieved to define Resin Infusion oil.

Strain Resin Infusion oil through cheese cloth collect said strained resin oil to define Strained resin oil.

Kung Fu GU Final composition:
4 oz. Strained infusion oil
4 oz. Strained Resin oil
1.6 oz Beeswax
2 oz. Menthol crystal
1 tsp. Wintergreen essential oil
1 tsp. Birch essential oil
1 tsp. Lavender essential oil
1 tsp. Cajuput essential oil
1 tsp. Clove essential oil

Position the for mentioned Strained Infusion oil and Strained Resin oil and the beeswax in a cooking vessel and heat until beeswax is completely melted, Then Add the menthol crystal once the menthol crystal have deliquesce, remove from the heat and add the essential oils, pour The molten balm emollient in at least one container and aloud to cool to define Kung Fu GU circulatory, balm, emollient.

Kung Fu GU is applied directly to the skin over the area of discomfort or pain by gently massaging Said balm into the skin two to three time a day although the effective amount may vary as needed or desired.

In the case of bruises, strained or sprained tendons or ligaments gently integument the affected area with a thin layer of Kung Fu GU, loosely cover said area with and bandage or gauze to localize the coverage.

Additional application may be necessary to achieve the desired comfort level.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those described in the specification are intended to be encompassed by the present invention.

Therefore since numerous modifications and changes will readily occur to those skilled in the art, it is not desired to limit the invention to the exact construction and operation described, and accordingly, all suitable modifications and equivalents may be resorted to, falling within the scope of the invention.

What is claimed:

1. Kung Fu GU a topical balm circulatory, emollient that helps accelerate the healing of bruises, and if used in a timely manner will help to prevent the manifestation of a bruise. As well as reduces swelling and pain of strained or sprained tendon and ligaments, as well as increases circulation further more expediting the healing process. Said balm comprising of Grape seed oil, Bees wax Rhubarb, Dandelion, Phellodendron, Safflower, Gardenia fruit, Skullcaps, Arnica Montana, Comfrey, Devil's claw Frankincense, Myrrh, Dragon's Blood, Menthol crystals Wintergreen oil, Birch oil, Lavender oil, Cajuput oil, Clove oil.

2. A method for making Kung Fu GU balm, circulatory, emollient comprised of Grape seed oil, Bees wax, Rhubarb, Phellodendron, Safflower, Dandelion, Gardenia fruit, Skullcaps, Arnica Montana, Comfrey, Devil's claw, Frankincense, Myrrh, Dragon's Blood, Menthol crystals, Wintergreen oil, Birch oil, Lavender oil, Cajuput oil, Clove oil.
